(19) United States
(12) Patent Application Publication
(10) Pub. No.: US 2008/0279914 A1
(43) Pub. Date: Nov. 13, 2008

(54) Transdermal composition comprising piroxicam-inorganic material complex and patch system comprising the same

(76) Inventors: Jae Hoon Jo, Daejeon (KR); Eun Mi Lee, Daejeon (KR); Yang Su Han, Kyungki-do (KR); Gee Young Jung, Seoul (KR)

Correspondence Address: GIBBONS P.C., ONE GATEWAY CENTER, NEWARK, NJ, 07102, US

(21) Appl. No.: 11/595,246

(22) Filed: Nov. 9, 2006

(30) Foreign Application Priority Data
Mar. 6, 2006 (KR) 2006-21071

(51) Int. Cl. A61K 9/70; A61K 31/5415

(52) U.S. Cl. 424/448; 514/226.5

(57) ABSTRACT

There is disclosed a transdermal composition comprising from about 0.1 to 25% by weight of a piroxicam-inorganic material complex composed of a swellable clay and piroxicam intercalated between layers thereof, from about 50 to 80% by weight of an adhesive polymeric material, and from about 0.1 to 25% by weight of an absorption enhancer. There is further disclosed a transdermal patch system comprising the subject transdermal composition. In the transdermal composition, piroxicam is dispersed on a molecular level between layers of a clay material. As a result, the dispersability, stability and solubility of the piroxicam are improved, the recrystallization of the piroxicam is prevented, and excellent absorption properties and skin permeability of the piroxicam are achieved. Therefore, excellent anti-inflammatory and analgesic effects are exhibited, little or no skin irritation is caused, thereby preventing occurrence of side effects, and the active ingredient is continuously administered by a transdermal route.

### RELATED APPLICATIONS

The present application is based on, and claims priority from, Korean Application Number 2006-21071, filed Mar. 6, 2006, the disclosure of which is incorporated herein by reference in its entirety.

### BACKGROUND OF THE INVENTION

1. Field of the Invention

The present invention relates to a transdermal composition comprising a piroxicam-inorganic material complex and a patch system for transdermal administration of piroxicam using the transdermal composition. More specifically, the present invention relates to a transdermal composition comprising a piroxicam-inorganic material complex in which the solubility and stability of the piroxicam are improved and the recrystallization of the piroxicam is prevented, and a patch system for transdermal administration of piroxicam using the transdermal composition.

2. Description of the Related Art

Many pharmacologically effective and physiologically active substances suffer from poor stability, as well as toxicity and side effects despite their superior beneficial effects. In addition, these substances may have limitations in use due to one or more of the following problems: first, they may have very low solubility, resulting in low bioavailability; second, they may have a bitter or pungent taste, making them suitable for oral administration; and finally, they may not be readily deliverable to particular tissues and sites.

For example, for delivery of highly pharmacologically active, poorly soluble or insoluble drugs to tissues and sites, the drugs should be rapidly dissolved upon oral or transdermal administration so that the they can be efficiently absorbed into the body. In proportion to this absorption, the blood concentrations of the drugs are increased and the thus-desired efficacy is exerted. Accordingly, low-solubility drugs cannot sufficiently exert their therapeutic effect in the body.

A number of methods have been proposed to effectively utilize active ingredients that necessitate improvements in the stability, safety, solubility, delivery characteristics, and the like. Some suggestions have been introduced to overcome these prior art problems. Representative examples include methods for preparing salts of active ingredients, methods for preparing complexes or derivatives by combining active ingredients with polymeric compounds or ligands, methods for encapsulating active ingredients with polymeric materials, methods for forming micelles of active ingredients using surfactants, methods for preparing solid dispersions of active ingredients, methods for forming complexes having a core-shell structure using active ingredients, methods for forming active ingredients into liposomes, methods for coating drug particles with biodegradable polymers, methods for producing nanoparticles of active ingredients by supercritical processes, methods for using inclusion compounds, e.g., cyclodextrin, and methods for adsorbing active ingredients to porous inorganic powders.

For example, pharmaceutical compositions comprising a piroxicam-cyclodextrin complex are known in the art, see for example, Korean Patent Laid-open No. 2003-0041577, which describes a solid dispersion of cyclodextrin and piroxicam. European Patent Application No. 0 848 936 A2 describes systems for transdermal administration of piroxicam using a copolymer. Korean Patent Laid-open No. 2003-0069373 describes the production of a tablet containing an L-arginine-piroxicam complex; and U.S. Pat. No. 4,233,299 describes water-soluble salts using piroxicam, meglumine, and glucamin. Korean Patent Laid-open No. 2000-0013593 describes a composition for transdermal administration of piroxicam and a water-solubilizing agent wherein the water-solubilizing agent contains at least one group selected from ammonium, amine, pyridine and pyrrolidine groups; Korean Patent Laid-open No. 1998-031601 describes a method for dissolving a poorly soluble drug in a microemulsion using a polyoxyethylene-based material; European Patent No. 0 529 123 describes a system for improving the bioavailability of piroxicam using a polymer gel formulation; and European Patent Application No. 88301417.7 and U.S. Pat. No. 4,867,985 describe the controlled release of piroxicam using a cellulosic copolymer.

On the other hand, synthetic and natural clay materials, which are types of inorganic materials, can be effectively utilized to achieve the desired properties, including stability, of active substances. For example, Korean Patent Laid-open No. 94-0019301 discloses an enteric coating composition prepared by adding at least one highly swellable inorganic material selected from zeolite, bentonite and highly swellable clay to a benzimidazole derivative.

U.S. Pat. No. 5,719,197 and WO 2002/102390 disclose that the use of clay in delivery systems of transdermal drugs improves the adhesiveness in transdermal formulations without reducing the delivery rate of a parent drugs/prodrug; WO 2003/059263 explains improvements in rheological properties, such as viscosity, flow characteristics and lubricity, of ophthalmic and otic pharmaceutical compositions by the addition of synthetic smectite clays; U.S. Pat. No. 6,177,480 describes the use of a synthetic clay material (Laponite™) as a wetting agent for contact lenses, which assists in the removal of lipid deposits from contact lenses by surfactants; U.S. Pat. No. 4,605,621 describes a method of preparing an enzyme-organoclay complex in an aqueous dispersion; U.S. Pat. No. 4,810,501 teaches the control of the sustained-release properties of a physiologically active substance, e.g., a drug, vitamin or mineral, by combining the active substance with an ionic particulate material, e.g., kaolin or silicate, and an ionically neutral polymer in an aqueous dispersion; U.S. Pat. No. 6,180,378 discloses an improvement in physiological activity by immobilizing a bioactive protein, such as an enzyme, using a layered silicate and an organosilicon compound in a solution; WO 2004/009120 A1 discloses a hybrid of a poorly soluble drug and a clay in a solution; and U.S. Pat. No. 5,840,336 teaches the use of a hydrated calcium silicate for improving the sustained-release properties of a hydrophilic drug by uniformly dispersing the drug in a hydrophobic polymer phase.

Piroxicam, a non-steroidal anti-inflammatory and analgesic drug, has been used to effectively treat acute pain and acute gout of rheumatoid arthritis, osteoarthritis, and musculoskeletal disorders. Piroxicam is generally administered orally and exerts potent therapeutic effects upon oral administration, but is known to cause side effects, such as gastrointestinal disorders and peptic ulcers.

Orally administered piroxicam is metabolized through the first-pass route. It is known that less than 5% of the drug is discharged unchanged in the urine and the activity of the metabolites is extremely weak. In contrast, the use of a patch-type system for transdermal delivery of piroxicam avoids the first-pass effects and enables continuous administration of a constant amount of piroxicam in a simple manner while substantially preventing occurrence of side effects in the body.

Some methods for transdermal administration of piroxicam have been attempted by including the active ingredient (i.e. piroxicam) at a high concentration in a base or using an absorption enhancer to increase the absorption of the active ingredient. According to these methods, however, skin irritation may be caused and recrystallization of the drug may occur due to the presence of an excess of the drug, leading to a reduction in the skin permeation of the drug and a decrease in its efficacy. In accordance with the present invention, a system for transdermal administration of piroxicam is provided that overcomes these disadvantages.

### SUMMARY OF THE INVENTION

In accordance with the present invention, a transdermal composition comprising a piroxicam-inorganic material complex is provided in which the stability, safety, dissolution properties, solubility, dispersibility, flow characteristics and transdermal permeability of the piroxicam as an active ingredient are improved and the recrystallization of the piroxicam is prevented. The patch system for transdermal administration of the invention affords the continuous transdermal administration of the piroxicam.

In accordance with one aspect of the present invention, there is provided a composition for transdermal administration of piroxicam which comprises 0.1-25% by weight of a piroxicam-inorganic material complex composed of piroxicam intercalated between layers of swellable clay, 50-80% by weight of an adhesive polymeric material, and 0.1-25% by weight of an absorption enhancer. The patch system for transdermal administration of piroxicam in accordance with the present invention utilized this transdermal composition.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing a patch system according to the present invention;

FIG. 2 is a graph showing the results of stability tests for patches produced in Examples 4 and 6 in Experimental Example 2; and

FIG. 3 is a graph comparing the concentrations of piroxicam in plasma of rabbits using a commercially available patch product and patches of the present invention prepared in Experimental Example 3.

### DESCRIPTION OF THE PREFERRED EMBODIMENTS

The present inventors have earnestly and intensively conducted research to increase the physical stability and prevent recrystallization of piroxicam, an anti-inflammatory and analgesic pharmaceutical, upon transdermal administration, and as a result, have found that the use of an intercalated piroxicam-inorganic material complex and a particular absorption enhancer contributes to improvements in the stability, safety, dissolution properties, solubility, dispersibility, flow characteristics and transdermal permeability of the piroxicam as well as preventing its recrystallization.

The piroxicam-inorganic material complex is formed by intercalating piroxicam as a pharmaceutically active ingredient between lattice layers of a swellable clay material. Intercalation is a well known process in which chemicals are inserted into interlayer spaces of layered materials, such as clays. An intercalated product in accordance with the present invention and recognized teachings in the art, is a layered material having a second material in the spaces between the layers. There are numerous articles in the literature describing various intercalated products and techniques. An example of a detailed discussion of intercalation can be found in Deng et al. Journal of Colloid and Interface Science, 250, 379-393, 2002. There is no particular restriction as to the type of the intercalated piroxicam-inorganic material complex used in the present invention. Likewise, the piroxicam-inorganic material complex may be formed by recognized processes, including both solution and dry processes.

The piroxicam-inorganic material complex is formed by solution process according to the following procedure. First, solvent molecules are coordinated to interlayer cations of a clay material to cause an intercalation, and as a result, the expansion, i.e. swelling, of lattice layers of the clay occurs. Due to the presence of the interlayer cations and swellability, which are inherent characteristics of clay, various intercalation reactions are induced. Then, an organic substance is intercalated between the lattice layers of the clay to form an organo-clay complex. This intercalation mainly occurs through an ion exchange reaction between the interlayer cations and organic cations, for example, alkyl ammonium ions.

According to a typical solution process, a suitable clay is dispersed in water, an organic solvent, or a mixture thereof to induce the swelling of lattice layers of the clay as a result of an intercalation of the water or solvent molecules, thereby extending the basal spacing of the lattice layers of the clay, and then a solution of an active ingredient is added thereto to induce an intercalation reaction of the active ingredient into the clay.

According to the dry process, an active ingredient is intercalated between layers of clay in the absence of a solvent or in the presence of a small amount of a solvent. For example, an active ingredient and a two-dimensional, swellable clay having interlayer cations are fed into a pulverizer, where the active ingredient and the clay are pulverized and mixed together, so that the active ingredient is intercalated between layers of the clay to form a complex. Since the dry process allows insoluble or poorly soluble active ingredients, such as piroxicam, to be distributed on a molecular level and enables the formation of complexes in high yields, it is particularly preferred for the formation of piroxicam-inorganic material complexes in accordance with the present invention.

In the dry process, the piroxicam-inorganic material complex can be formed by pulverizing and mixing piroxicam and an inorganic material (i.e. a clay material) at ambient pressure and temperature, under heat, vacuum or pressure, or under heat and pressure by pelletizing the mixture. The pulverization and mixing can be performed for from about 5 minutes to about 48 hours. Below 5 minutes, piroxicam is not sufficiently intercalated between the lattice layers of the clay. Above 48 hours, there is a risk that the piroxicam molecules may reaggregate, thereby causing recrystallization of the drug. Continued mixing and pulverization over 48 hours is also undesirable due to the increased energy consumption and inherent costs.

While the pulverization and mixing are performed at ambient temperature and pressure, additional heat treatment may be subsequently performed, if desired. The pulverization and mixing, or the optional heat treatment, are carried out at a temperature of 300° C. or less, and preferably at temperatures from about 40° to 300° C. The reason for the limitation of the temperature to a maximum of 300° C. is to prevent piroxicam being thermally degraded and denatured. In addition, in those instances where undesired release of heat takes place during the pulverization and mixing, the particle size of piroxicam is very large, or the mixing ratio between piroxicam and the clay material is extremely low, it is preferred to add a slight amount of a solvent before the pulverization and mixing. In this case, the amount of the added solvent by weight is less than total weight of the mixture of the clay material and the piroxicam.

As the solvent, there can be used, without intended limitation, water, an organic solvent or a mixed solvent thereof. Specific examples of suitable organic solvents include those having a boiling point at which they can be evaporated during the pulverization and heating, for example, methanol, ethanol, acetone, acetylacetone and methylene chloride. There are various methods of forming intercalated complexes known in the art. For example, the piroxicam-inorganic material complex can be formed by the method described in Korean Patent Laid-open No. 2006-0044200.

As the inorganic material of the piroxicam-inorganic material complex, there can be used a swellable clay material that has a two-dimensional layered structure and exhibits a peculiar interlayer reactivity. Each layer of the two-dimensional layered structure has a thickness of 1 to 2 nm. The clay material may be a synthetic or natural clay.

When an active ingredient, i.e. piroxicam, is introduced between layers of the layered clay material, the active ingredient dispersed on a molecular level is uniformly encapsulated with nanometer-sized inorganic lattice layers of the clay material. In addition, the active ingredient molecules introduced between the layers of the clay material having a layered structure are stabilized by various interactions, for example, electrostatic interaction between the negatively charged inorganic lattice layers and the positively charged active ingredient through ion exchange, interlayer complexation of cations present between the inorganic lattice layers and the active ingredient molecules, hydrogen bonding between water or hydroxyl groups present between the inorganic lattice layers and hydrogen atoms present in the active ingredient, interaction by transfer of charges from the inorganic lattice layers to the active ingredient or from the active ingredient to the inorganic lattice layers, van der Walls interaction between the hydrophobic interlayer material and the hydrophobic active ingredient, and electric dipole interaction between the polar inorganic lattice layers and the polar active ingredient. Specifically, the swellable clay material may be a silicate and preferably an alumina silicate. More specifically, the swellable clay material may be a smectite clay having an ion exchange capacity. The smectite clay may be selected from the group consisting of montmorillonite, bentonite, hectorite, fluorohectorite, nontronite, beidellite, saponite, vermiculite, rectorite, fluoromica, swellable mica, and mixtures thereof.

The shape, size and cation exchange capacity (CEC) of the clay are not particularly restricted. For efficient reaction, it is preferred the clay have a shape of a sphere, a needle or a plate, a size of 10 μm or less, and a cation exchange capacity of 60-200 meq./100 g. The interlayer cations of the clay compound are also not particularly restricted, and are preferably Na⁺, K⁺, Li⁺, NH₄⁺, H⁺, Ag⁺, Ca²⁺, Mg²⁺, Co²⁺, Ni²⁺, Fe²⁺, Zn²⁺, Mn²⁺, Cu²⁺, Al³⁺ and Fe³⁺, and more preferably H⁺, Ca²⁺, Mg²⁺, Ni²⁺, Zn²⁺, Cu²⁺, Al³⁺ and Fe³⁺.

The interlayer and/or surface of the clay compound may be modified before use in the formation of the piroxicam-inorganic material complex. This modification can be conducted using amines, ammonium salts, organosilicon compounds, polynuclear metal compounds, metal oxide nanoparticles, and the like. Specifically, the surface and/or interlayer of the clay compound can be previously modified with the following compounds: amines, e.g., tetradecylamine, hexadecylamine, octadecylamine, and salts thereof; alkyl and aromatic quaternary ammonium compounds, e.g., dimethyldistearyl ammonium, trimethyltetradecyl ammonium, trimethylhexadecyl ammonium, trimethyloctadecyl ammonium, benzyltrimethyl ammonium, benzyltriethyl ammonium, and phenyltrimethyl ammonium; and other cationic surfactants and polymers. The interlayer-modified clay material advantageously decreases steric hindrance arising from intercalation of the piroxicam so that the intercalation reaction with the piroxicam can be efficiently induced.

The surface and/or interlayer of the clay material can be wholly or partially modified with an organosilicon compound, e.g., tetramethoxysilicate, tetraethoxysilcate, propyltrimethoxysilicate, octyltriethoxysilicate, or aminosilane. Modification of the clay material with an organosilicon compound increases the efficiency of the intercalation interaction thereof with piroxicam.

The interlayer of the clay material may be crosslinked with a cationic polynuclear metal complex or metal oxide nanoparticles. For example, a representative cationic polynuclear metal compound, such as an iron hydroxide cluster [Feₓ(OH)_{y}]^{z+} or an aluminum cluster {[Al₁₃O₄(OH)₂₄]⁷⁺}, is ion-exchanged with sodium ions present between the clay layers to form what is called a “crosslinked clay”. The polynuclear metal compound is preferably a material having cationic properties, for example, a hydroxide with metal ions, an inorganic hydroxide cluster compound, or an organometallic cluster compound, and examples thereof include Al₁₃O₄(OH)₂₄(H₂O)₁₂⁷⁺, Si(acetylacetone)⁺, [Bi₆(OH)₁₂]⁶⁺, Fe₃(OH)₄⁵⁺, Zr₄(OH)₈.H₂O⁸⁺, (TiO)₂(OH)₈⁴⁺, and [Fe₃O(CH₃COO)₆]⁺. Examples of suitable inorganic hydroxide clusters include aluminum hydroxide clusters, e.g., Al₁₃O₄(OH)₂₄⁷⁺, zirconium hydroxide clusters, e.g., Zr₄(OH)₁₄²⁺, titanium hydroxide clusters, e.g., (TiO)₂(OH)₈⁴⁺, iron hydroxide clusters, e.g., Feₓ(OH)_{y}^{z+}, composite metal hydroxide clusters, e.g., [SiO₂—TiO₂]^{z+}, [{Al₁₃O₄(OH)₍₂₄₋ₙ₎}—{OSi(OH)₃}ₙ]⁷⁺ and [SiO₂—Feₓ(OH)_{y}]^{z+}, and organometallic clusters, e.g., [Fe₃(OCOCH₃)₆]¹⁺. In the formulae, x, y, z and n represent the degree of polymerization of the corresponding metals. The degree of polymerization, which is an inherent characteristic of the metals, may be varied according to various conditions, such as pH, temperature and concentration. These conditions can be suitably selected within the ranges that are commonly known to those skilled in the art.

The metal oxide nanoparticles may be nanoparticles of at least one metal oxide selected from the group consisting of Al₂O₃, TiO₂, SiO₂, Fe₂O₃, and ZrO₂. In the case where the interlayer of the clay is modified through the intercalation reaction of polynuclear metal compound or the metal oxide nanoparticles, the spacing of the lattice layers of the clay is extended to 0.5-10 nm. Accordingly, the steric hindrance of the active ingredient is reduced upon a dry solid reaction, thus facilitating the intercalation of the active ingredient. In addition, the specific surface area and the porosity of the clay are increased, which increases the amount of the active ingredient contained in the clay. Furthermore, the stability of the active ingredient can be further enhanced, and the sustained-release properties of the active ingredient can be more accurately controlled.

If necessary, the clay material is heat-treated before use in the formation of the piroxicam-inorganic material complex so that the content of the water or solvent in the interlayer of the clay material can be controlled. However, the heat-treatment is not necessarily required.

The mixing ratio between the clay material and the piroxicam for the formation of the piroxicam-inorganic material complex can be determined depending on the desired final content of the piroxicam in the piroxicam-inorganic material complex. In accordance with the present invention, the weight ratio of the swellable clay to the piroxicam is preferably 1:0.1˜10 and more preferably 1:1˜3. If the weight ratio is less than 1:0.1, the advantages arising from the intercalation of the piroxicam cannot be substantially achieved. Meanwhile, if the weight ratio is more than 1:10, a relatively large amount of the piroxicam remains unreacted and thus the various advantages of the intercalation-type piroxicam-inorganic material complex are not substantially realized.

The piroxicam-inorganic material complex formed on a molecular level exhibits the following properties. First, since the piroxicam is distributed on a molecular level, the solubility of the piroxicam in water or an organic solvent is increased exponentially. Second, since the piroxicam interacts with the inorganic lattice layers on a molecular level, the piroxicam molecules do not aggregate and thus, recrystallization of the piroxicam is prevented. Third, the inorganic lattice layers surrounding the piroxicam protect the piroxicam against degradation, denaturation and destruction caused by ambient conditions, i.e. heat, light, oxygen, humidity and bases, leading to an improvement in the stability of the piroxicam. Fourth, since the piroxicam encapsulated with the inorganic lattice layers is slowly released by various factors, such as ion exchange and concentration difference, the toxicity, side effects and irritation resulting from the fast release and direct contact to the skin of the piroxicam are considerably reduced. Fifth, since the piroxicam is slowly released from the inorganic lattice layers, the pharmacological effects of the piroxicam can be consistently provided. Sixth, since the surface characteristics of the piroxicam particles are modified by the inorganic lattice layers, the dispersability of the piroxicam in water, particularly solvent systems, can be relatively freely controlled.

In use, the pharmaceutically active ingredient molecules distributed within the inorganic matrix are replaced by externally supplied ionic materials and are slowly dissolved. The replacement of the drug molecules with cationic materials in various states present on the skin or within skin tissues is a basic mechanism in the dissolution of the drug in the skin. Particularly, sweat (NaCl) and skin wastes (e.g., organic substances, including organic acids) present in the skin induce the release of the drug.

Accordingly, the formulation of the piroxicam-inorganic material nano-complex into a patch system contributes to a marked improvement in the stability of the piroxicam and to the inhibition of recrystallization of piroxicam within the patch. In addition, highly-permeable preparations of the drug can be designed, although the drug is present at a low effective concentration in the preparations.

The present invention provides a transdermal composition comprising the piroxicam-inorganic material complex. The composition for transdermal administration of piroxicam according to the present invention comprises piroxicam as a pharmaceutically active ingredient with anti-inflammatory and analgesic activity, an absorption enhancer, an adhesive polymer, and the like. The piroxicam as a pharmaceutically active ingredient is provided in the form of the piroxicam-inorganic material complex in which the piroxicam is intercalated between layers of an inorganic material.

The piroxicam-inorganic material complex is present in an amount of from about 0.1 to 25% by weight in the transdermal composition, which is used to form a matrix layer of a patch system. When the complex is present in an amount of less than about 0.1% by weight, the anti-inflammatory and analgesic effects are negligible. Conversely, when the complex is present in an amount exceeding about 25% by weight, the relative amounts of the other components of the composition, particularly the adhesive polymeric material and the absorption enhancer, are reduced thereby unduly increasing the viscosity of the composition resulting in difficulties in handling the composition and a decrease in the transdermal permeability of the piroxicam contained therein.

The absorption enhancer is utilized to enhance the skin permeation of the active ingredient, and may be at least one compound selected from the group consisting of cetearyl ethylhexanoate, isopropyl myristate, polyethylene glycol-8 glyceryl linoleate, and polypropylene glycol monolaurate. The absorption enhancer is present in an amount of from about 0.1 to 25% by weight in the transdermal composition, which is used to form a matrix layer of a patch system. When the content of the absorption enhancer is less than about 0.1% by weight, the skin permeation of the active ingredient is not enhanced. Conversely, when the content of the absorption enhancer is greater than about 25% by weight, the adhesiveness of the transdermal composition is reduced and it is difficult to maintain the shape of the matrix layer in a patch system.

The absorption enhancer markedly improves the skin absorption rate of the piroxicam, compared to other absorption enhancers generally used in the art. By the use of the absorption enhancer, the skin permeation and absorption of the active ingredient are maximized in a patch using the transdermal composition.

The adhesive polymeric material serves to transport and store the active ingredient, and plays a role in imparting good diffusion properties in a contact area with the skin while the patch system is adhered to the skin to firmly affix the matrix layer to the skin.

The adhesive polymeric material may be present in an amount of from about 50 to 80% by weight in the transdermal composition. When the content of the adhesive polymer is less than about 50% by weight, the transport and storage capacity of the pharmaceutically active ingredient are reduced, the adhesiveness of the thermal composition is deteriorated, and the diffusion properties of the active ingredient are poor, causing a marked deterioration in use efficiency when the patch is attached to the skin. Conversely, when the content of the adhesive polymer is greater than about 80% by weight, the content of the pharmaceutically active ingredient is relatively low, causing a reduction in transdermal administration effects, and the content of the absorption enhancer is relatively low, causing poor administration effects.

The adhesive polymeric material is a medicinally acceptable pressure-sensitive adhesive ingredient. As the adhesive polymeric material, an aqueous or organic solvent-based material can be used. The adhesive polymeric material is not particularly restricted, and can be suitably selected among adhesive polymeric materials that can be generally used to form matrix layers of patches in the art. Examples of suitable adhesive polymeric materials include commercially available products, in which an acrylic resin is blended with an organic solvent material such as an acrylic, self-curing pressure sensitive adhesive (PSA) (DURO-TAK 2677, National Starch & Chemical Products).

The transdermal composition may further comprise at least one of the additive selected from solubilizing agents, dissolution assistants, dispersants, absorption auxiliaries, and other pharmaceutically active ingredients that are routinely used in the art. These additives can be suitably selected within the ranges that are commonly used in the art taking into consideration the reactivity with and affinity for the active ingredient (i.e. piroxicam).

The present invention also provides a patch system for transdermal administration of piroxicam using the transdermal composition. When the patch system of the present invention is attached to the skin, piroxicam is transdermally administered from a matrix layer including the piroxicam-inorganic material complex.

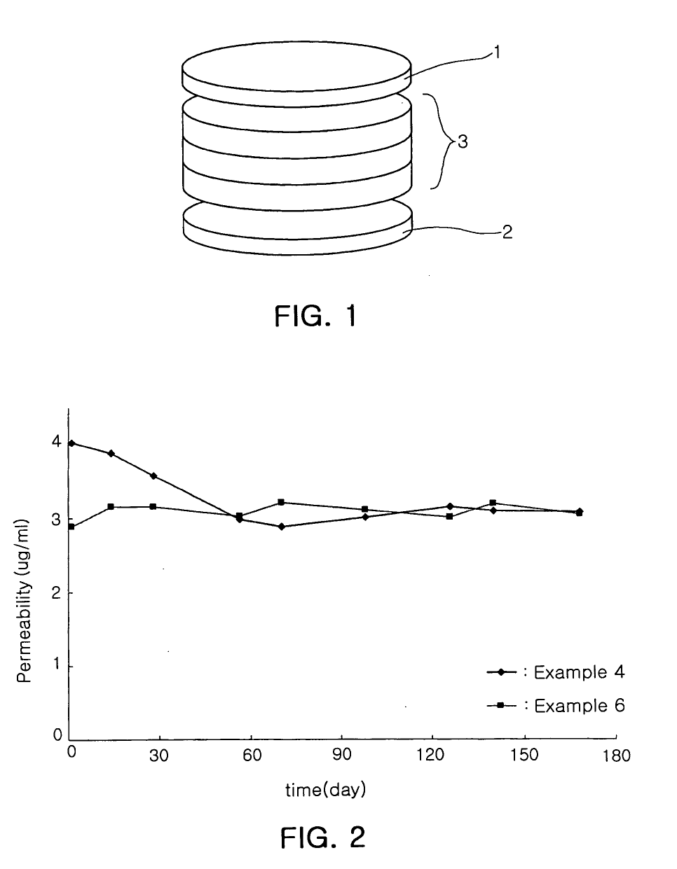

FIG. 1 shows the patch system for transdermal administration of piroxicam according to the present invention. As shown in FIG. 1, the patch system of the present invention is comprised of a backing layer 1, a release layer 2 and a matrix layer 3 interposed between the backing layer and the release layer. The matrix layer 3 is made of the transdermal composition of the present invention.

Conventional piroxicam-containing patches must contain piroxicam at a high concentration within a range that does not cause precipitation of a crystal in order to ensure an appropriate degree of permeation of the drug. In contrast, the patch system of the present invention exhibits a high degree of permeation of the drug even at a low concentration because of the piroxicam-inorganic material nanocomplex included therein.

If required, the matrix layer 3 may be formed into a monolayer or a multilayer consisting of two or more layers. When the matrix layer 3 is comprised of multilayers, the release rates of the active ingredient may vary in the respective layers. An illustration of such multilayer construction is shown in FIG. 1 wherein the matrix layer 3 consists of three layers.

The non-permeable backing layer 1 can be made of materials through which the active ingredient of the matrix layer 3 cannot permeate. The materials for the backing layer 1 are not restricted and includes all such materials commonly used in the art. For example, the backing layer may be made of a polyurethane or polyethylene film.

The patch system of the present invention is produced by forming the matrix layer 3 into a monolayer or multilayer on the backing layer 1, and attaching the release layer 2 to the matrix layer. The release layer 2 can be made of materials commonly used in the art. For example, the release layer 2 is made of, without any particular limitation, a fluoropolymer-coated polyester film (3M-Scotchpak 1022) or a silicone foil.

When the patch system of the present invention is attached to the skin, a therapeutically effective amount of the piroxicam is released from the piroxicam-inorganic material complex included in the matrix layer 3 and is transdermally administered.

Since the piroxicam is present in the form of the piroxicam-inorganic material complex included in the matrix layer of the patch system according to the present invention, the dispersibility, stability, solubility of the piroxicam are improved, the recrystallization of piroxicam is prevented, and excellent absorption properties and skin permeability of the piroxicam are achieved by the action of the absorption enhancer.

Therefore, even if piroxicam is present in a small amount, excellent anti-inflammatory and analgesic effects are exhibited, little or no skin irritation is caused, thereby preventing occurrence of side effects, and piroxicam is continuously administered by a transdermal route while the patch system is attached to the skin.

The present invention will now be described in more detail with reference to the following examples. However, these examples are not intended to limit the present invention in any way.

### Example 1

### Formation of Piroxicam-Inorganic Material Complex by a Dry Process

A magnesium silicate (Magnabrite F, AMCOL, USA) having an average diameter of about 1.2 μm, a pharmaceutically acceptable inorganic layered silicate, was dried at 120° C. for 12 hours. A total of 50 g of piroxicam was added to 150 g of the silicate, and then 200 g of acetone was added thereto to dissolve the piroxicam. The resulting mixture was introduced into a 1 liter-ball mill and zirconia balls (300 g) were filled therein. Thereafter, the reactants were homogeneously mixed together in the ball mill at 350 rpm for one hour. After mixing, nitrogen gas preheated to 100° C. was fed into the ball mill at a rate of 1 L/min. through one end of the ball mill to allow the piroxicam molecules to intercalate reaction between layers of the layered silicate. The reaction time was adjusted to 2 hours. After completion of the reaction, the zirconia balls were removed using a 100-mesh standard screen to obtain a piroxicam-inorganic material complex powder that showed a characteristic X-ray diffraction line (˜4° at 20) due to the intercalation of the piroxicam molecules, and had a piroxicam content of 25 wt %.

### Example 2

### Formation of Piroxicam-Inorganic Material Complex by a Dry Process

Example 1 was repeated utilizing 10 g of piroxicam and 100 g of the magnesium silicate. The resulting piroxicam-inorganic material complex powder showed a characteristic X-ray diffraction line (˜3.8° at 2θ) due to the intercalation of the piroxicam molecules, and had a piroxicam content of 50 wt %.

### Example 3

### Formation of Piroxicam-Inorganic Material Complex by a Solution Process

A total of 50 g of a magnesium silicate (Magnabrite F, AMCOL, USA) as utilized in Example 1 was dispersed in a mixed solvent of distilled water (2.5 L) and ethanol (2 L) in an Erlenmeyer flask. Separately, 16.6 g of piroxicam was dissolved in 500 ml of ethanol. The clay dispersion was stirred in a thermostatic bath at 60° C. for 30 minutes, and then the piroxicam solution was slowly added thereto. The pH of the mixture was adjusted to 1.5 using 170˜200 ml of 2N HCl. After completion of the titration, the Erlenmeyer flask was closed using a silicone stopcock. The reactants were stirred for 2 hours and washed three times with distilled water/ethanol (50/50(v/v). The obtained sample was dried in an electric convection oven at 100° C. for 12 hours to obtain a piroxicam-inorganic material complex.

### Example 4

### Production of a Transdermal Patch

The piroxicam-inorganic material complex prepared in Example 1, a dissolution assistant, a dispersant and an absorption enhancer were sufficiently mixed together in accordance with the composition indicated in the following table, and then a solution of Kollidon SR and DuroTak 2677 (45:55 (w/w)) as adhesive polymers in ethyl acetate was added thereto. After the mixture was stirred for 10 minutes, gas bubbles were removed from the mixture.

The mixture was applied to a thickness of 1.3 mm to a release layer (3M-Scotchpak 1022™) and dried at 70° C. for 24 hours to completely remove the volatile solvents. A polyurethane film was attached thereto. Thereafter, the resulting structure was cut to a size of 20 cm² to obtain a final product.

| Ingredient | Percent by Weight |
|---|---|
| Piroxicam-inorganic material complex, | 15 |
| Example 1, 25 wt % | |
| Crodamol CAP⁽¹⁾ | 10 |
| Eutanol GM⁽²⁾ | 2 |
| Polysorbate 80⁽²⁾ | 4 |
| Oleic acid⁽²⁾ | 1 |
| Adhesive polymers | 68 |

⁽¹⁾Crodamol CAP ™, a mixture of cetearyl ethylhexanoate and isopropyl myristate, was used as the absorption enhancer.
⁽²⁾Eutanol GM ™ is 2-Octyl Dodecyl Myristate. Eutanol GM ™, Polysorbate 80 and oleic acid function as both dissolution assistant and dispersant.

### Example 5

### Production of Patch

The piroxicam-inorganic material complex prepared in Example 2, a dissolution assistant, a dispersant and an absorption enhancer were sufficiently mixed together in accordance with the composition indicated in the following table, and then a solution of Kollidon SR and DuroTak 2677 (45:55 (w/w)) as adhesive polymers in ethyl acetate was added thereto. After the mixture was stirred for 10 minutes, gas bubbles were removed from the mixture.

The mixture was applied to a thickness of 1.3 mm to a release layer (3M-Scotchpak 1022™) and dried at 70° C. for 24 hours to completely remove the volatile solvents. A polyurethane film was attached thereto. Thereafter, the resulting structure was cut to a size of 20 cm² to obtain a final product.

| Ingredient | Percent by Weight | |
|---|---|---|
| Piroxicam-inorganic material | 15 | wt % |
| complex, 50 wt % | | |
| Crodamol CAP | 10 | wt % |
| Eutanol GM | 2 | wt % |
| Polysorbate 80 | 4 | wt % |
| Oleic acid | 1 | wt % |
| Adhesive polymers | 68 | wt % |

### Example 6

### Production of Patch

The piroxicam-inorganic material complex prepared in Example 1, a dissolution assistant, a dispersant and an absorption enhancer were sufficiently mixed together in accordance with the composition indicated in the following table, and then a solution of Kollidon SR and DuroTak 2677 (45:55 (w/w)) as adhesive polymers in ethyl acetate was added thereto. After the mixture was stirred for 10 minutes, gas bubbles were removed from the mixture.

The mixture was applied to a thickness of 1.5 mm to a release layer (3M-Scotchpak 1022™) and dried at 70° C. for 24 hours to completely remove the volatile solvents. A polyurethane film was attached thereto. Thereafter, the resulting structure was cut to a size of 20 cm² to obtain a final product.

| Ingredient | Percent by Weight | |
|---|---|---|
| Piroxicam-inorganic material complex | 15 | wt % |
| Example 1, 25 wt % | | |
| Lauroglycol 90⁽³⁾ | 11 | wt % |
| Eutanol GM | 1 | wt % |
| Polysorbate 80 | 2 | wt % |
| Adhesive polymers | 71 | wt % |

⁽³⁾Lauroglycol 90 ™ is propylene glycol monolaurate, and was used as the absorption enhancer.

### Example 7

### Production of Patch

The piroxicam-inorganic material complex prepared in Example 2, a dissolution assistant, a dispersant and an absorption enhancer were sufficiently mixed together in accordance with the composition indicated in the following table, and then a solution of Kollidon SR and DuroTak 2677 (45:55 (w/w)) as adhesive polymers in ethyl acetate was added thereto. After the mixture was stirred for 10 minutes, gas bubbles were removed from the mixture.

The mixture was applied to a thickness of 1.5 mm to a release layer (3M-Scotchpak 1022™) and dried at 70° C. for 24 hours to completely remove the volatile solvents. A polyurethane film was attached thereto. Thereafter, the resulting structure was cut to a size of 20 cm² to obtain a final product.

| Ingredient | Percent by Weight | |
|---|---|---|
| Piroxicam-inorganic material complex | 15 | wt % |
| Example 2, 50 wt % | | |
| Lauroglycol 90 | 11 | wt % |
| Eutanol GM | 1 | wt % |
| Polysorbate 80 | 2 | wt % |
| Adhesive polymers | 71 | wt % |

### Example 8

### Production of Patch

The piroxicam-inorganic material complex prepared in Example 1, a dissolution assistant, a dispersant and an absorption enhancer were sufficiently mixed together in accordance with the composition indicated in the following table, and then a solution of Kollidon SR and DuroTak 2677 (45:55 (w/w)) as adhesive polymers in ethyl acetate was added thereto. After the mixture was stirred for 10 minutes, gas bubbles were removed from the mixture.

The mixture was applied to a thickness of 1.5 mm to a release layer (3M-Scotchpak 1022™) and dried at 70° C. for 24 hours to completely remove the volatile solvents. A polyurethane film was attached thereto. Thereafter, the resulting structure was cut to a size of 20 cm² to obtain a final product.

| Ingredient | Percent by Weight | |
|---|---|---|
| Piroxicam-inorganic material complex | 15 | wt % |
| Example 1, 25 wt % | | |
| Propylene glycol-8 glyceryl linoleate⁽⁴⁾ | 10 | wt % |
| Eutanol GM | 1 | wt % |
| Polysorbate 80 | 1 | wt % |
| Oleic acid | 2 | wt % |
| Adhesive polymers | 71 | wt % |

⁽⁴⁾Propylene glycol-8 glyceryl linoleate was used as the absorption enhancer.

### Example 9

### Production of Patch

The piroxicam-inorganic material complex prepared in Example 2, a dissolution assistant, a dispersant and an absorption enhancer were sufficiently mixed together in accordance with the composition indicated in the following table, and then a solution of Kollidon SR and DuroTak 2677 (45:55 (w/w)) as adhesive polymers in ethyl acetate was added thereto. After the mixture was stirred for 10 minutes, gas bubbles were removed from the mixture.

The mixture was applied to a thickness of 1.5 mm to a release layer (3M-Scotchpak 1022™) and dried at 70° C. for 24 hours to completely remove the volatile solvents. A polyurethane film was attached thereto. Thereafter, the resulting structure was cut to a size of 20 cm² to obtain a final product.

| Ingredient | Percent by Weight | |
|---|---|---|
| Piroxicam-inorganic material complex | 15 | wt % |
| Example 2, 50 wt % | | |
| Propylene glycol-8 glyceryl linoleate | 10 | wt % |
| Eutanol GM | 1 | wt % |
| Polysorbate 80 | 1 | wt % |
| Oleic acid | 2 | wt % |
| Adhesive polymers | 71 | wt % |

### Experimental Example 1

### Skin Absorption Tests

The skin absorption tests were conducted on the piroxicam patches using skins of hairless mice. Each of the skins was excised just before experiment, and fixed in the middle part of a Franz-type diffusion cell so that the horny layer of the skin was directed upward. A 30% polyethylene glycol solution was added to a receptor, and stirring was continued at a constant rate of 600 rpm using a magnetic stirrer while maintaining at 37° C. At 2, 4, 6, 12, 18 and 24 hour intervals after each of the patches produced in Examples 4 and 6 was attached to the skin, predetermined amounts of the solution contained in the receptor were sampled and a fresh buffer solution was replenished in the same amounts as the sampled amounts. The concentration of the piroxicam in the samples was measured by high-performance liquid chromatography (HPLC) under the following analytical conditions.

Column: Zorbox Eclipse C₁₈ 5 μm

Mobile phase: Distilled water/acetonitrile/acetic acid=497.5/500/2.5 (v/v/v)

Flow rate: 1.0 ml/min.

Detector: UV 360 nm

The contents and skin absorption rates of the drug in a commercially available piroxicam patch product (Trast™, SK Chemicals, Korea) and the patches produced in Examples 4 and 6 were measured, and the results are shown in Table 1 below.

TABLE 1

| Patch products | Trast ™ | Example 4 | Example 6 |
|---|---|---|---|
| Piroxicam content in | 7.5 wt % | 3.75 wt % | 3.75 wt % |
| patches | | | |
| Flux (μg/cm²/h) | 1.58 ± 0.13 | 3.23 ± 0.65 | 2.87 ± 0.37 |

As can be seen from the results in Table 1, the permeability of piroxicam in the patches produced in Examples 4 and 6 was two or more times higher than that in the Trast™ patches. These results indicate that the permeability of piroxicam in the patches is increased due to the formation of the piroxicam-inorganic material complex, even if the piroxicam is present in approximately half the amount.

### Experimental Example 2

### Stability Tests

The patches produced in Examples 4 and 6 were sealed and stored in a light-shielded state under accelerating conditions at 40° C. and 75% relative humidity. For 6 months of storage, the occurrence of recrytallization of the piroxicam in the patches was checked and the permeability of the piroxicam was tested. The results are shown in FIG. 2. The graph of FIG. 2 demonstrates that the patches using the transdermal compositions of the present invention exhibited stable permeability of piroxicam even after six months of storage.

### Experimental Example 3

### Blood Concentration Measurement

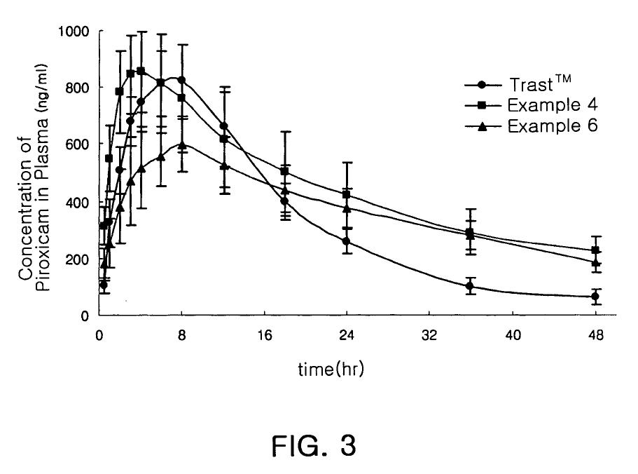

The blood concentrations of piroxicam in male rabbits (NZW) weighing 2-3 kg were measured using a commercially available piroxicam patch product (Trast™) and the patches produced in Examples 4 and 6, all patches having the same area (20 cm²). First, hairs on the back of the animals were removed one day before the measurement, and each of the patches was attached to the back. Blood samples were obtained from the animals at intervals over 48 hours. The obtained blood samples were centrifuged to obtain plasma samples. The analysis of the plasma samples was conducted by high-performance liquid chromatography, and the analytical results are shown in FIG. 3. The graph of FIG. 3 shows that the patches produced in Examples 4 and 6 demonstrated superior transdermal permeability of piroxicam with the passage of time as compared to commercially available patch product (Trast™).

As apparent from the above description, the transdermal composition and the patch system of the present invention comprise a piroxicam-inorganic material complex and a particular absorption enhancer. Since piroxicam is dispersed on a molecular level between layers of a clay material in the transdermal compositions of the invention, the dispersability, stability and solubility of the piroxicam are improved, the recrystallization of the piroxicam is prevented, and excellent absorption properties and skin permeability of the piroxicam are achieved. Therefore, even if the active ingredient (i.e. piroxicam) is used in a small amount, excellent anti-inflammatory and analgesic effects are exhibited, little or no skin irritation is caused, thereby preventing occurrence of side effects, and the active ingredient is continuously administered by a transdermal route while the patch system is attached to the skin.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A composition for the transdermal administration of piroxicam, comprising: from about 0.1 to 25% by weight of a piroxicam-inorganic material complex composed of piroxicam intercalated between layers of swellable clay; from about 50 to 80% by weight of an adhesive polymeric material; and from about 0.1 to 25% by weight of an absorption enhancer.

2. The composition according to claim 1, wherein the swellable clay is a natural or synthetic clay.

3. The composition according to claim 1, wherein the swellable clay is a silicate.

4. The composition according to claim 3, wherein the swellable clay is a smectite clay material.

5. The composition according to claim 4, wherein the smectite clay material is at least one material selected from the group consisting of montmorillonite, bentonite, hectorite, fluorohectorite, nontronite, beidellite, saponite, vermiculite, rectorite, fluoromica, and swellable mica.

6. The composition according to claim 1, wherein the swellable clay has a shape of a sphere, a needle or a plate, an average size of 10 μm or less, and a cation exchange capacity of 60-200 meq./100 g.

7. The composition according to claim 1, wherein the swellable clay contains at least one interlayer cationic species selected from the group consisting of Na⁺, K⁺, Li⁺, NH₄⁺, H⁺, Ag⁺, Ca²⁺, Mg²⁺, Co²⁺, Ni²⁺, Fe²⁺Zn²⁺, Mn²⁺, Cu²⁺, Al³⁺ and Fe³⁺.

8. The composition according to claim 1, wherein the swellable clay has at least one of an interlayer and a surface modified with one or more members selected from the group consisting of an amine, an alkyl and aromatic quaternary ammonium compound, an organosilicon compound, a polynuclear metal compound and metal oxide nanoparticles.

9. The composition according to claim 8, wherein the swellable clay has at least one of an interlayer and a surface modified with at least one compound selected from the group consisting of tetradecylamine, hexadecylamine, octadecylamine, and salts thereof, dimethyldistearyl ammonium, trimethyltetradecyl ammonium, trimethylhexadecyl ammonium, trimethyloctadecyl ammonium, benzyltrimethyl ammonium, benzyltriethyl ammonium, and phenyltrimethyl ammonium.

10. The composition according to claim 8, wherein the organosilicon compound is at least one compound selected from the group consisting of tetramethoxysilicate, tetraethoxysilcate, propyltrimethoxysilicate, octyltriethoxysilicate, and aminosilane.

11. The composition according to claim 8, wherein the polynuclear metal compound has cationic properties and is selected from the group consisting of a hydroxide with metal ions, an organometallic cluster compound and an inorganic hydroxide cluster compound.

12. The composition according to claim 11, wherein the polynuclear metal compound is at least one compound selected from the group consisting of: Al₁₃O₄(OH)₂₄(H₂O)₁₂⁷⁺; Si(acetylacetone)₃⁺; [Zr₄(OH)₈.H₂O]⁸⁺; [(TiO)₈(OH)₁₂]⁴⁺; [Bi₆(OH)₁₂]⁶⁺; [Fe₃(OH)₄]⁵⁺ and [Fe₃O(CH₃COO)₆]⁺.

13. The composition according to claim 8, wherein the metal oxide nanoparticles are nanoparticles of at least one metal oxide selected from the group consisting of Al₂O₃, TiO₂, SiO₂, Fe₂O₃, and ZrO₂.

14. The composition according to claim 1, wherein the absorption enhancer is at least one compound selected from the group consisting of cetearyl ethylhexanoate, isopropyl myristate, polyethylene glycol-8 glyceryl linoleate, and polypropylene glycol monolaurate.

15. The composition according to claim 1, wherein the adhesive polymeric material is an acrylic resin.

16. A patch system for transdermal administration of piroxicam, wherein piroxicam is present therein as a transdermal composition according to claim 1.

17. The patch system according to claim 16, wherein the patch system includes a matrix layer made comprising said transdermal composition, wherein said matrix layer is formed into a monolayer or multilayers.

18. The composition according to claim 1, wherein the piroxicam-inorganic material complex is formed by a mixing piroxicam and said swellable clay by a process selected from the group consisting of: a solution process wherein piroxicam is dissolved in a suitable solvent and mixed with said swellable clay; a dry process wherein piroxicam and said swellable clay are pulverized and mixed without a solvent, or in the presence of an amount of solvent not exceeding their total weight; with heating at a temperature not exceeding 300° C.

19. The composition according to claim 18, wherein the swellable clay has at least one of an interlayer and a surface modified with an amine, an alkyl and aromatic ammonium compound, an organosilicon compound, a polynuclear metal compound, or metal oxide nanoparticles prior to mixing with piroxicam.

20. The composition according to claim 18, wherein the piroxicam-inorganic material complex is formed by mixing the swellable clay and piroxicam in a weight ratio of 1:0.1˜10.

21. The composition according to claim 20, wherein the piroxicam-inorganic material complex is formed by mixing the swellable clay and piroxicam in a weight ratio of 1:1˜3.
